(19) United States
(12) Patent Application Publication
(10) Pub. No.: US 2020/0090792 A1
(43) Pub. Date: Mar. 19, 2020

(54) HANDS-FREE SPEECH-BASED NATURAL LANGUAGE PROCESSING COMPUTERIZED CLINICAL DECISION SUPPORT SYSTEM DESIGNED FOR VETERINARY PROFESSIONALS

(71) Applicant: Ethos Veterinary Health, LLC, Woburn, MA (US)

(72) Inventors: Patrick M. Welch, Ipswich, MA (US); Ken Hubbell, Raleigh - Durham, NC (US); Jeff Johnson, Seattle, WA (US)

(21) Appl. No.: 16/364,537

(22) Filed: Mar. 26, 2019

Related U.S. Application Data
(60) Provisional application No. 62/648,056, filed on Mar. 26, 2018.

(51) Int. Cl. G16H 10/60; G10L 15/30; G10L 15/22; G10L 15/18; H04R 1/02; H04R 3/00; G06F 3/16; A61B 5/00

(52) U.S. Cl. CPC G16H 10/60; G10L 15/30; G10L 15/22; G10L 15/1815; H04R 1/028; H04R 2420/07; G06F 3/167; A61B 5/749; G10L 2015/223; H04R 2499/11; H04R 2499/15; H04R 3/00

(57) ABSTRACT

A hands-free speech-based natural language processing clinical decision support (CDS) system configured to operate in conjunction with a stationary or mobile base device speech system to receive voice commands from a user. A dialog may be conducted with the user in multiple turns, where each turn comprises user speech and a speech response by the speech system. The user speech in any given dialog turn may be provided from the base device. This speech system dialog is directed by a computer program finite state engine. Rule data for the state engine is retrieved from an internet cloud database by a computer program function and is applied to the speech dialog system in order to prompt the user for specific information. This user supplied information is used as inputs to the computer program that in turn refine the speech dialog and requests for additional information. Once all information required to make the CDS recommendation have been received, the computer program applies an algorithm to generate a set of recommendations from which the user can select the best option for their patient.

### BACKGROUND OF THE INVENTION

### Field of the Invention

The present invention relates to the use of a hands free, speech based, computerized natural language processing system to provide veterinary professionals clinical decision support in a clinical environment.

### Description of the Background

In various occupations, the need for hands-free assistance for Clinical Decision Support (“CDS”) is a practical means for a single person to accomplish the combined task of keeping their hands focused on the specific task or activity while simultaneously accessing the information they need to complete the task or activity or make decisions regarding the task or activity.

As the processing power available to devices and associated support services continues to increase, it has become practical to interact with users in new ways. In particular, it has become practical to interact with users through two-way speech dialogs, in which a user instructs a system by voice and the system responds by speech.

It is now practical to develop and deploy computer software to interface with these voice-based systems in a way specifically designed to extract from the user the information required to drive algorithms to provide accurate, real-time clinical decision support.

### SUMMARY OF THE INVENTION

A hands-free speech-based natural language processing clinical decision support system (knowledge and patient specific data and recommendations intelligently filtered to improve patient care and medical outcomes) configured to operate in conjunction with a stationary or mobile base device speech system to receive voice commands from a user. A user may direct speech to the base device. In order to direct speech to the base device, the user first speaks a keyword. A dialog may be conducted with the user in multiple turns, where each turn comprises user speech and a computer-generated audio speech response by the speech system. In addition, or as an alternative, the system response may be rendered in text on a display for the user to view. The user speech in any given dialog turn may be provided to the base device. The system response speech in any given dialog turn may be provided from the base device. This speech system dialog model is directed by a computer program finite state engine. Rule data for the state engine is retrieved from an internet cloud database by a computer program function and is applied to the speech dialog system in order to prompt the user for specific information. This user supplied information is used as inputs to the computer program that in turn refine the speech dialog and requests for additional information. Once all information required to make the CDS recommendation have been received, the computer program applies an algorithm to generate a set of recommendations from which the user can select the best option for their patient. During the speech dialog, each user interaction is stored in two separate cloud databases. The first database stores all user responses as the user progresses through the speech dialog. The user may start and stop a dialog at any time, returning to the point of departure upon return. The second database stores the user activity and decision process information to be used to provide data for reporting and analytics. User activity may include no response over a given period of time; requests to edit previous responses; and requests to exit the system prior to completion. The activity tracking records the time of the activity in addition to the activity itself.

### BRIEF DESCRIPTION OF THE DRAWINGS

FIG. 1 shows the user, patient, system in a typical use setting.

FIG. 2 shows the system architecture.

FIG. 3a-3i shows the application of the rules engine to control the dialog flow between the system and the user and data exchange points with the database.

FIG. 4 shows the function for updating the patient American Society of Anesthesiologists (ASA) rating referenced throughout FIG. 3a-3i.

FIG. 5 shows the algorithm for the ASA decision process.

Appendix I shows the dialog model for mapping input speech to functional intent.

Appendix II shows a sample speech request to be processed through the speech service and dialog model.

### DETAILED DESCRIPTION

A speech-based system may be configured to interact with a user through speech to receive instructions from the user and to provide information services for the user. The system may have a stationary or mobile base device which has a microphone for producing audio containing user speech. The user may give instructions to the system by directing speech to the base device.

Audio signals produced by the base device are provided to a speech service for automatic speech recognition (ASR) and natural language understanding (NLU) to determine and act upon user intents (e.g., instructions, responses, questions). The speech service is a combination of networked and non-networked computer programs running on a base hardware device and on an Internet distributed computer server that is configured to respond to user speech by sending data to custom computer program functions.

In order to fully determine a user's intent when speaking, the system may engage in a speech dialog with the user. A dialog comprises a sequence of dialog turns. Each dialog turn comprises a user utterance and may also include a system-generated audio speech reply. The following is an example of a speech dialog that may take place between a speech-based system and a user:

Turn 1: User: “Edit age.” System: “Is this patient between thirteen weeks and seven years old?”

Turn 2: User: “No.” System: “Is the patient less than thirteen weeks old?”

Turn 3: User: “No.” System: “This patient is greater than seven years old. How much does this patient weigh?”

A speech dialog may comprise any number of turns, each of which may use collected speech input from either the base device or the handheld device and corresponding response speech output deployed through the base device or the handheld device.

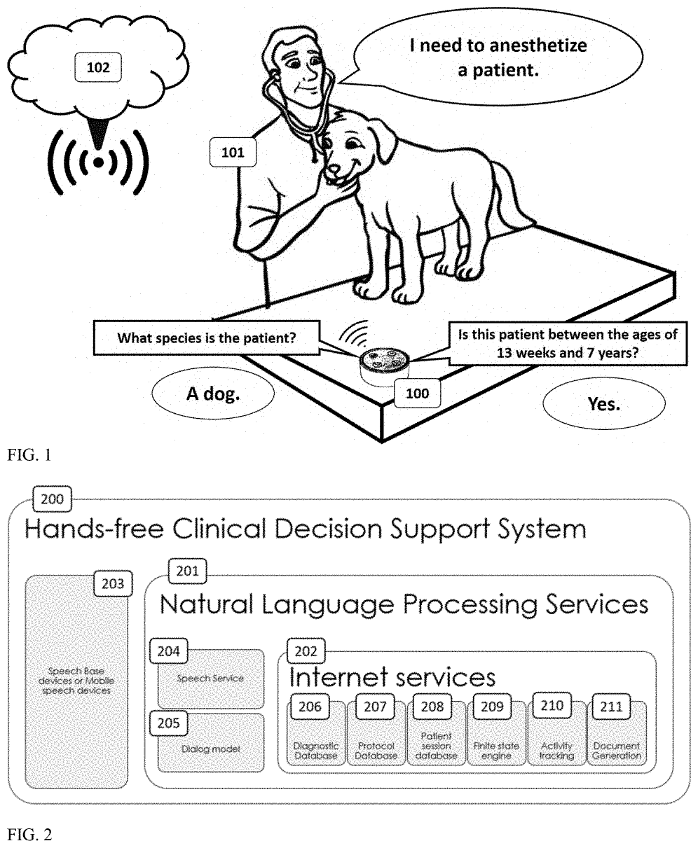

FIG. 1 shows an example speech-based system having a base device 100. The system may be implemented within an environment such as a room or an office, and a user 101 is shown as interacting with the system base device 100.

The base device 100 comprises a network-based or network-accessible speech interface device having one or more microphones, a speaker, and a network interface or other communications interface. The base device 100 is designed to be stationary and to operate from a fixed location, such as being placed on a stationary surface. The base device 100 may have omnidirectional microphone coverage and may be configured to produce an audio signal in response to a user utterance of a keyword.

The speech base device 100 includes a speech service 102 that receives real-time audio or speech information processed by the speech base device 100 in order to recognize user speech, to determine the meanings and intents of the speech, and to interface with the computer finite state engine in fulfillment of the meanings and intents. The speech service 102 also generates and provides speech for output by the base device 100.

The speech service 102 is part of a network-accessible computing platform that is maintained and accessible via the Internet. Network-accessible computing platforms such as this may be referred to using terms such as “on-demand computing”, “software as a service (SaaS)”, “platform computing”, “network-accessible platform”, “cloud services”, “data centers”, and so forth. Communications between the base device 100 and the service 102 may be implemented through various types of data communications networks, including local-area networks, wide-area networks, and/or the public Internet. Cellular and/or other wireless data communications technologies may also be used for communications. The speech service 102 may serve a large number of base devices and associated handheld devices, which may be located in the premises of many different users.

In FIG. 1, the user 101 is shown communicating with the speech service 102 by speaking in the direction toward the base device 100 while using their hands for other tasks. The speech service 102 may respond to the base device 100.

FIG. 2 shows the hands-free clinical decision support system (CDS) 200 as a diagram. The system 200 includes a natural language processing server 201 and a set of Internet services 202 for applying rules to assign protocols given a set of patient attributes provided by the user 101. 200 is the whole CDS that encompasses two sub systems: the speech processing sub system 201 and the database and custom finite state engine sub system 202. The speech base or mobile device 203 is the outer most part of the system and provides speech input and output. The Internet service for speech processing 204 works in combination with dialog model 205/APPENDIX I to control the response and request cycle of the dialog model. The Internet services sub system include the databases 206-208, the finite state engine 209, the activity tracking database 210, and the WIFI networked document generator application 211.

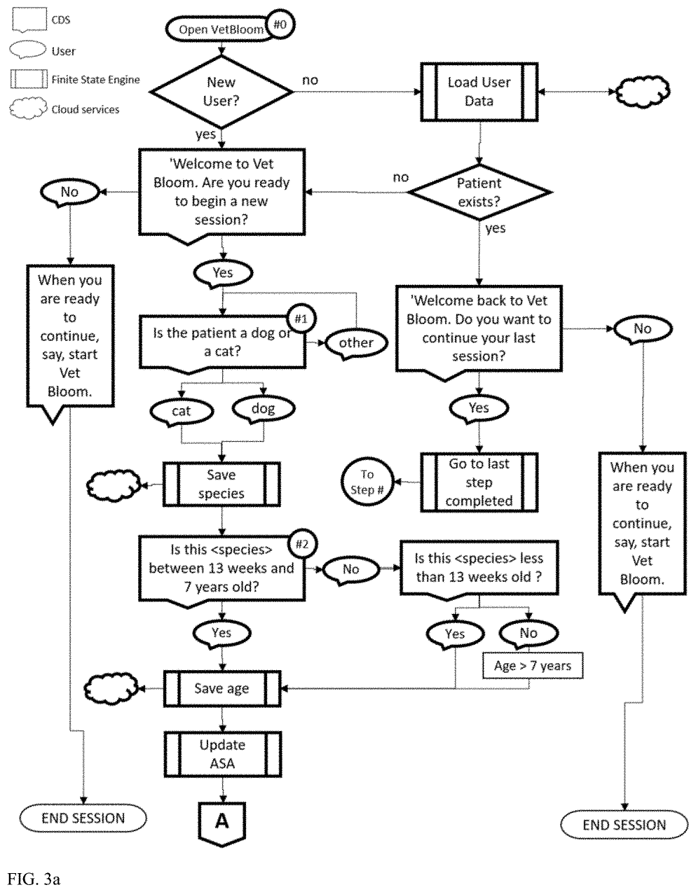

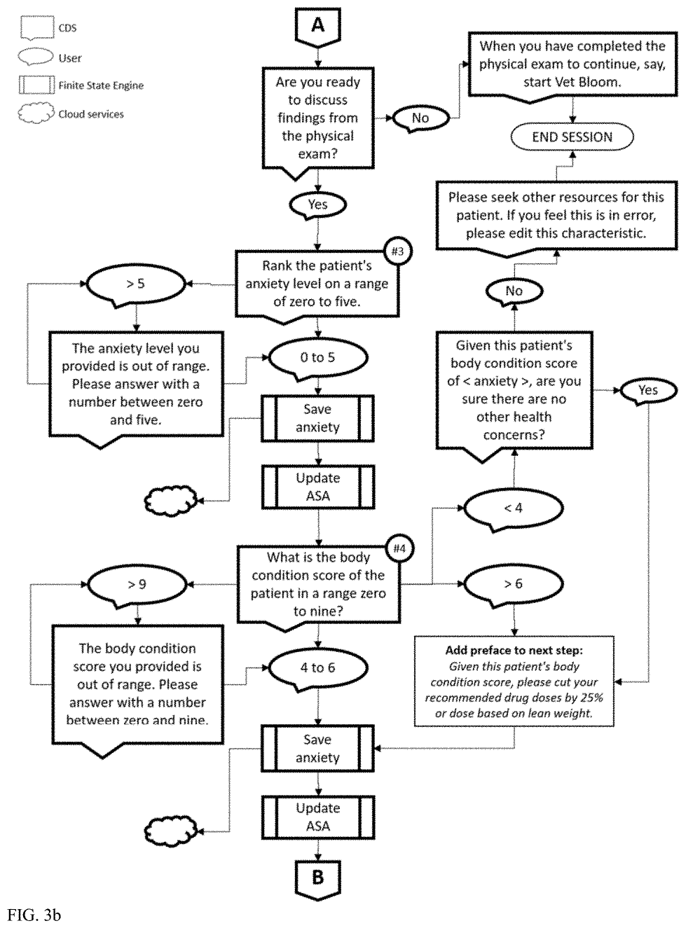

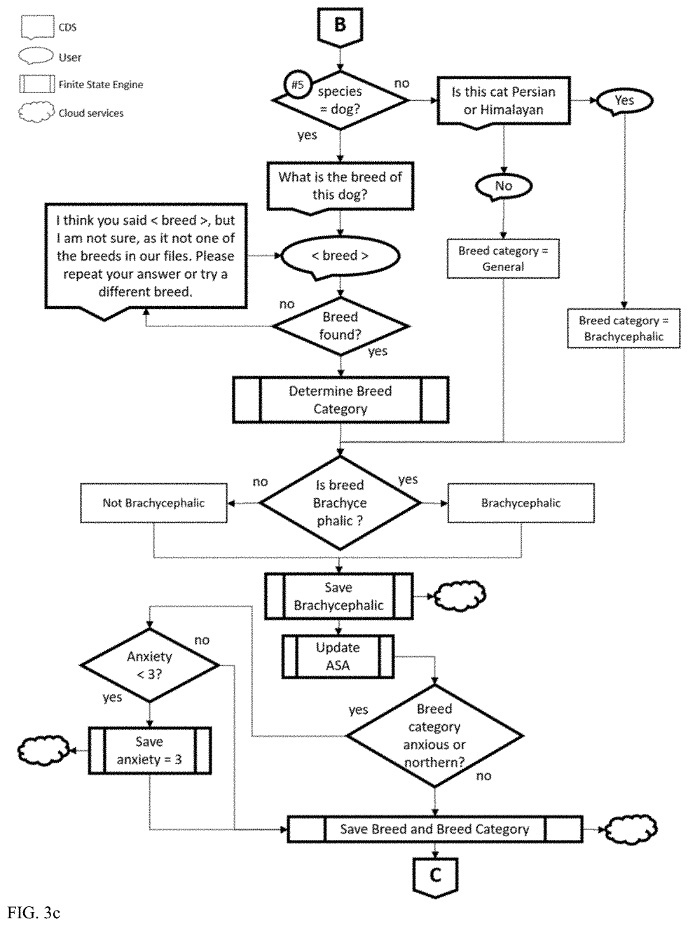

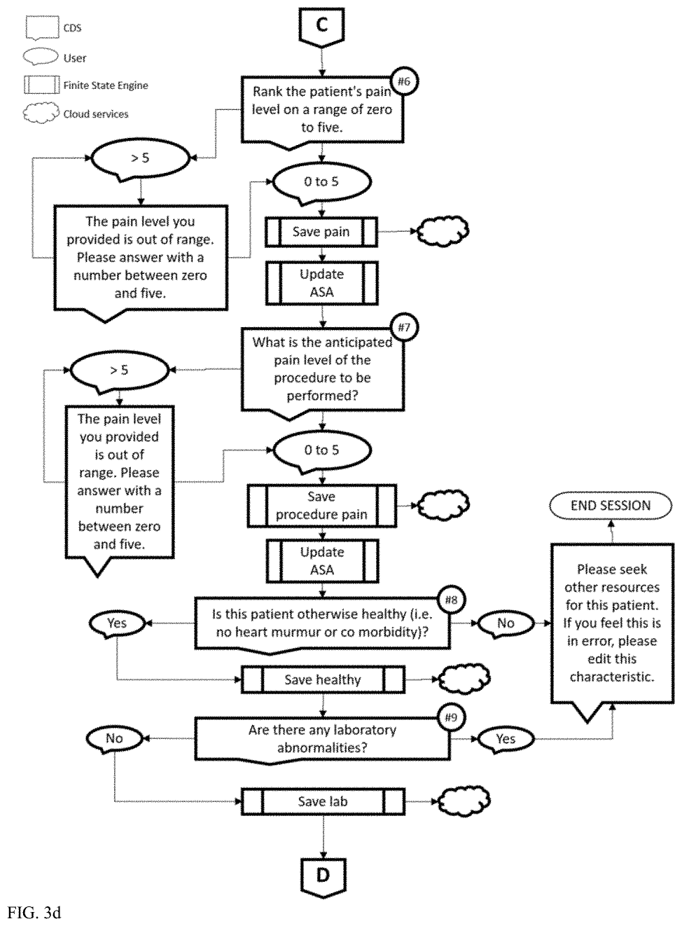

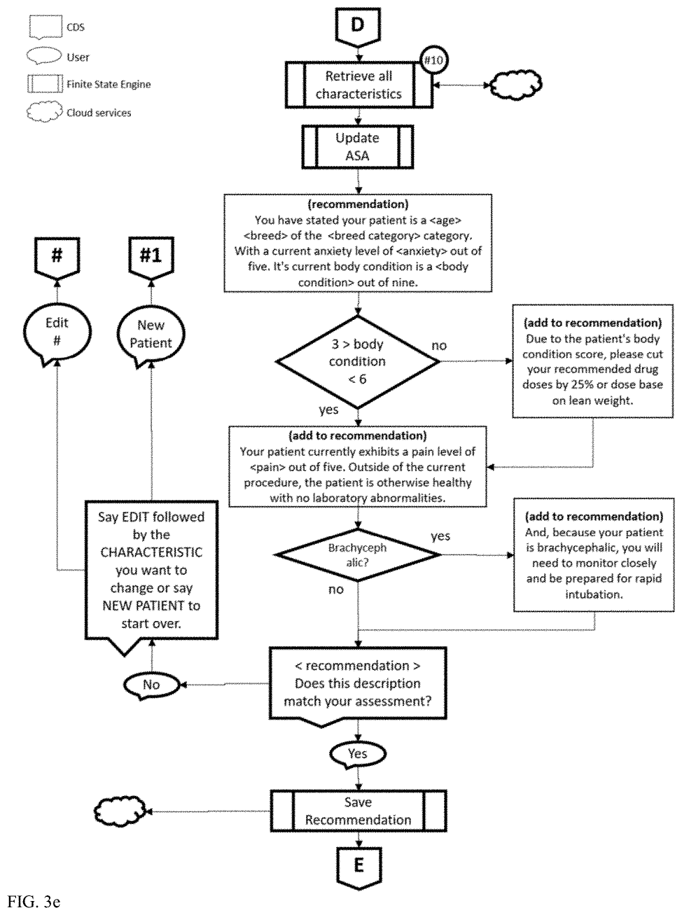

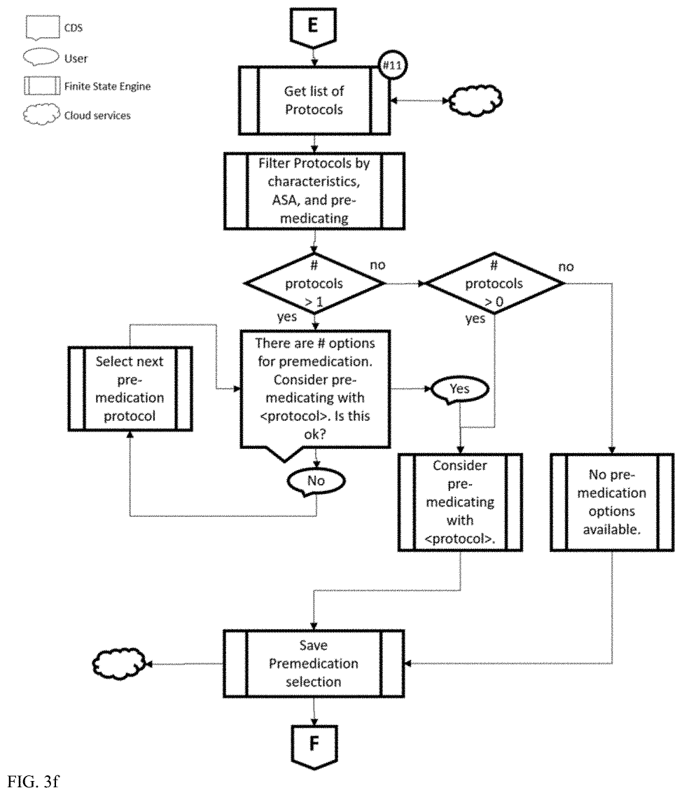

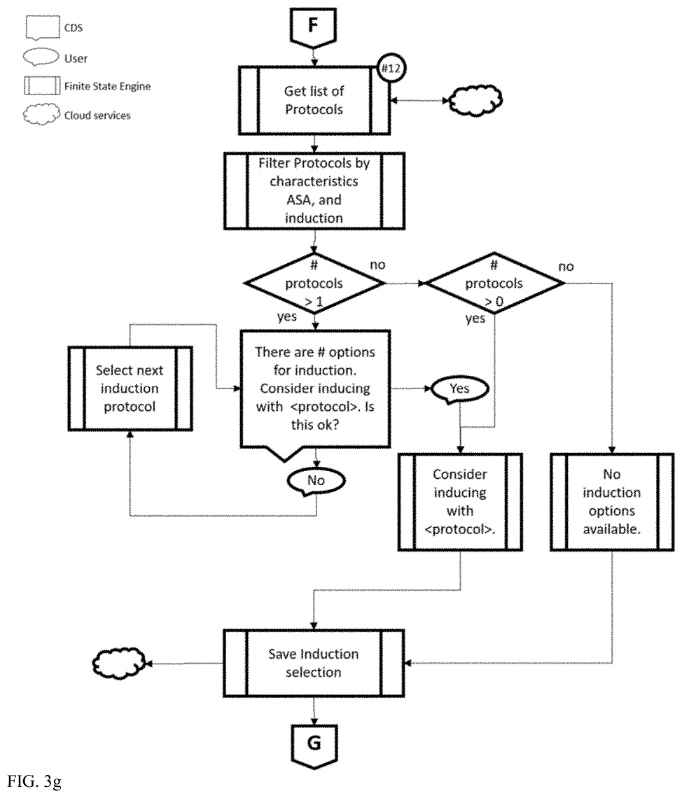

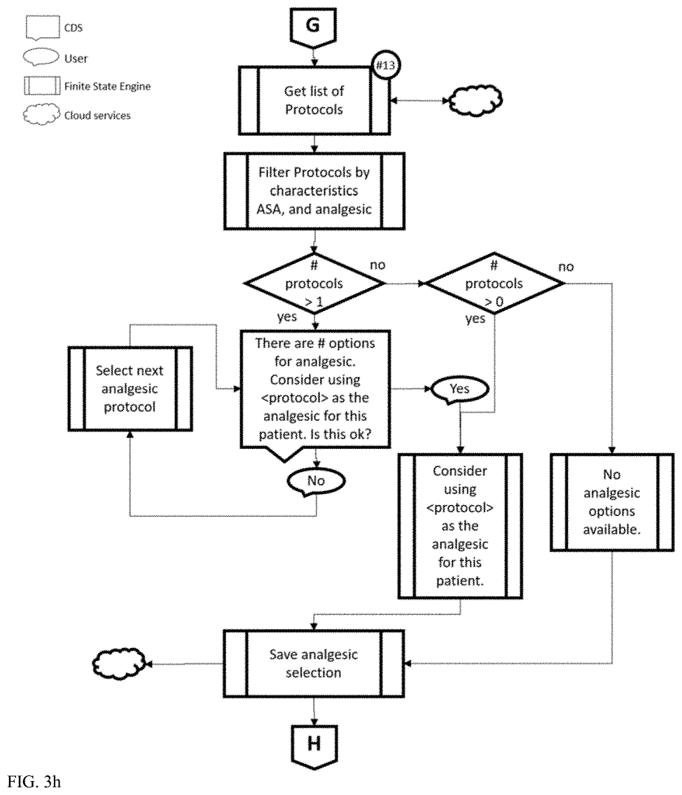

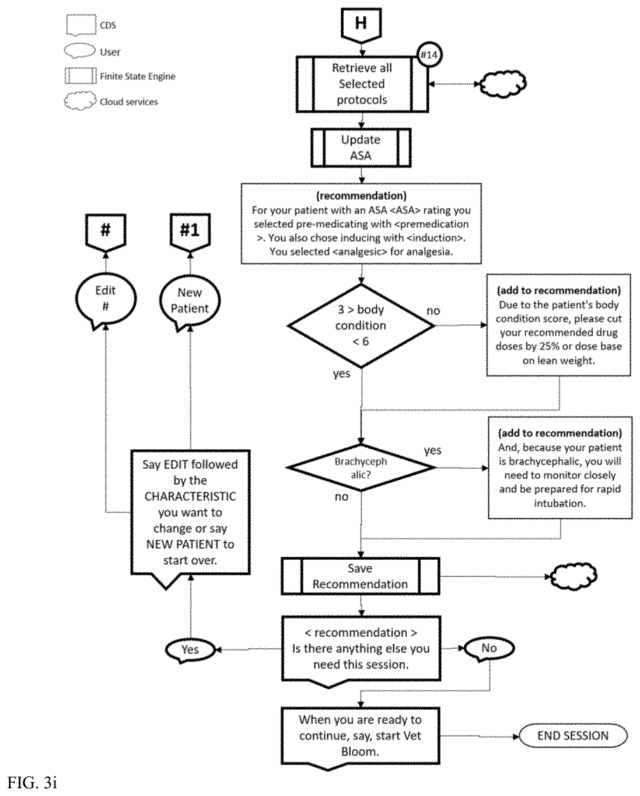

The CDS collects speech input from the networked speech device 203/100. The CDS runs the speech input through a speech service 204 and applies a dialog model 205/APPENDIX I to determine the action the user intended to implement. The dialog model APPENDIX I is used by the speech service 204 to map the user input speech to specific intents by direct word value or by associating the listed synonym of the expected word value. The dialog model 205/600 then sends the input speech data to the finite state engine in a formatted response APPENDIX II where it is processed FIG. 3a-i and then uses the speech service 204 to send a response/request output to the speech device 203 to play the audio for the user 101. The user 101 then replies with a patient session attribute that is sent back though the speech device 203 to the speech service 204 and dialog model 205. Each time the user 101 starts new or continues inputting data for a specific patient a patient session is created with a set of attributes associated with that patient. During the session, these attributes are configured by the user 101 by the user supplying inputs to the speech base device 100 in response to dialog requests from the speech service 102 based on the dialog model APPENDIX I. The dialog model 205/APPENDIX I makes a function call to the finite state engine 209/FIG. 3a-i that applies data from the diagnostic database 206 to determine where to apply the attribute to the patient database 208. The attribute decision activity is also saved to the activity tracking service 210. The user 101 can state a request through the speech base device 100 and speech service 204 to the dialog model 205 to edit patient attributes that have already been stored in the patient session database 208. This request sets the finite state engine 209 into edit mode and a request is generated though the dialog model 209 and output through the speech base device 100 for the user to supply a new value for the requested attribute. The user 101 can follow a similar process to request the dialog model 205 repeat the last request issued by the finite state engine 209. Once all attributes are input by the user 101, the finite state engine 209 requests the correct protocols from the protocol database 207 to respond to the speech service the recommendation for the patient. This response is sent to the speech device and the audio is played for the user 101. The final protocol recommendations are also saved to the patient session database 208 and the activity tracking service 210. The user 101 can also request a digital or print document of the protocols using the document generation service 211.

FIG. 3a-3i illustrates the dialog flow between the user and the CDS. The flowchart legend FIG. 3a-3i depicts the interaction of external computer program services hosted on Internet computer systems separate from the base device as driven by the dialog model APPENDIX I. CDS refers to the speech requests generated by the speech service 102 dialog model APPENDIX I. User refers to user 101 responses APPENDIX II to CDS requests output to the user through speech base device 100. The finite state engine refers to the finite state engine that applies a set of rules to the user input response to determine the correct action to execute next in the process FIG. 3a-i. Cloud services refers to all databases and other Internet services accessed by the system.

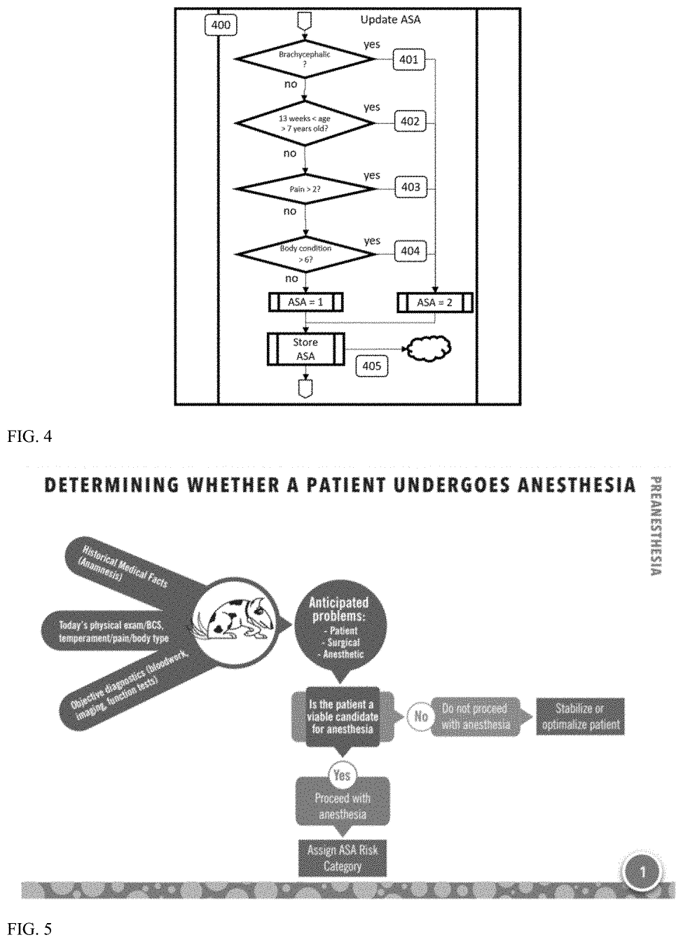

The process flow begins with FIG. 3a step #0 when the user opens the CDS by stating “Open Vet Bloom.” The finite state engine then validates the user and loads existing patient data if available. The CDS responds to the user in one of two ways. The user starts the protocol recommendation for either a new patient with no existing characteristic inputs or at the last attribute the user input for an existing patient. At each major step (#1-#13), the user is prompted to reply with an attribute or validation of a user decision. During the completion of each step, the attributes related to that step are processed by the finite state engine and, in FIG. 4, the patient ASA (American Society of Anesthesiologists) rating 400 is updated to reflect these characteristics in this order: Brachycephalic 401, Age 402, Pain 403, and Body Condition 404. The ASA and attributes are then saved to the patient session database and the activity to the activity tracking server 405. At any time, the user may request a prompt be repeated by the CDS. At any time, the user may request to edit a previously entered characteristic. At any time, the user may request to terminate the session. Once the user completes all of the attribute selection steps, in step #14, the CDS uses the rules state engine to apply the protocol database to determine the protocol recommendation for the patient. This protocol is then sent to the speech service 102 to be transmitted to the speech base device 100 where it is output as audio to the user 101.

While the claimed invention has been described in detail in connection with only a limited number of embodiments, it should be readily understood that the invention is not limited to such disclosed embodiments. Rather, the claimed invention can be modified to incorporate any number of variations, alterations, substitutions or equivalent arrangements not heretofore described, but which are commensurate with the spirit and scope of the invention. Additionally, while various embodiments of the claimed invention have been described, it is to be understood that aspects of the invention may include only some of the described embodiments. Accordingly, the claimed invention is not to be seen as limited by the foregoing description.

```
APPENDIX II
{
  “version”: “1.0”,
  “session”: {
    “new”: false,
    “sessionId”: “amzn1.echo-api.session.##########################################”,
    “application”: {
      “applicationId”: “amzn1.ask.skill.##########################################”
    },
    “attributes”: {
      “body_condition”: 5,
      “breed_category”: “canine giant breed”,
      “editmode”: true,
      “analgesic”: “a pure mu opioid, such as, Hydromorphone 0.1 milligram per kilogram,
intra muscular or intravenous”,
      “age_group”: 0,
      “STATE”: “BREED_STATE”,
      “premedication_note”: null,
      “breed”: “mastiff”,
      “continueflag”: false,
      “ASA”: 2,
      “anticipated_pain”: 5,
      “bookmark”: “BREED_STATE”,
      “editreturnpoint”: “FINAL_STATE”,
      “anxiety”: 4,
      “abnormal”: “no”,
      “dosingMultiplier”: 1,
      “species”: “dog”,
      “healthy”: “yes”,
      “breed_id”: “canine_giant_breed”,
      “current_pain”: 3,
      “inducing”: “Ketamine 1 milligram per kilogram, followed by Propofol, up to 4 milligram
per kilogram, titrated to effect”,
      “prescribe”: true,
      “premedication”: null,
      “brachycephalic”: true
    },
    “user”: {
      “userId”: “amzn1.ask.account.##########################################”
    }
  },
  “context”: {
    “AudioPlayer”: {
      “playerActivity”: “IDLE”
    },
    “Display”: {
      “token”: “”
    },
    “System”: {
      “application”: {
        “applicationId”: “amzn1.ask.skill.##########################################”
      },
      “user”: {
        “userId”: “amzn1.ask.account.##########################################”
      },
      “device”: {
        “deviceId”: “amzn1.ask.device.##########################################”,
        “supportedInterfaces”: {
          “AudioPlayer”: { },
          “Display”: {
            “templateVersion”: “1.0”,
            “markupVersion”: “1.0”
          }
        }
      },
      “apiEndpoint”: “https://api.amazonalexa.com”,
      “apiAccessToken”: “##########################################”
    }
  },
  “request”: {
      “type”: “IntentRequest”,
      “requestId”: “amzn1.echo-api.request.##########################################”,
      “timestamp”: “2018-03-11T22:35:53Z”,
      “locale”: “en-US”,
      “intent”: {
        “name”: “BreedIntent”,
        “confirmationStatus”: “NONE”,
        “slots”: {
          “species”: {
            “name”: “species”,
            “confirmationStatus”: “NONE”
          },
          “article”: {
            “name”: “article”,
            “confirmationStatus”: “NONE”
          },
          “breed”: {
            “name”: “breed”,
            “value”: “husky”,
            “resolutions”: {
              “resolutionsPerAuthority”: [
                {
                “authority”: “amzn1.er-authority.echo-
sdk.amzn1.ask.skill.##########################################.AMAZON.Animal”,
                “status”: {
                  “code”: “ER_SUCCESS_MATCH”
                },
                “values”: [
                  {
                    “value”: {
                      “name”: “canine northern breed”,
                      “id”: “canine_northern_breed”
                    }
                  }
                ]
              }
            ]
          },
          “confirmationStatus”: “NONE”
        }
      }
    }
  }
}
End of APPENDIX II
```

1. A hands-free speech-based natural language processing clinical decision support (CDS) system for use by a veterinary professional during a veterinary procedure while the user's hands are occupied or unable to access patient or pharmaceutical data without stopping the procedure, comprising: a stationary device or mobile device having a microphone and a speaker or earphone connected by wire or wirelessly; a natural language processing server programmed with computer software dialog model to interpret the raw voice data connected to said stationary device or mobile device via communications network; a patient information database with patient information connected to said stationary device or mobile device via communications network; a database with formulary rules to define protocols connected to a remote hosted computer application server via communications network; a patient session database to store patient session attributes while determining protocols connected to a remote hosted computer application server via communications network; a tracking database to track user actions and decisions connected to a remote hosted computer application server via communications network; an analytics system for analyzing user actions and decisions for audit, training and legal purposes connected to a remote hosted computer application server via communications network; interface for network interconnectivity to hospital information systems (HIS); interface for net interconnectivity to pharmaceutical inventory and purchasing to ensure availability of protocols before recommendation and to trigger ordering of pharmaceuticals when inventory runs low; a logic state rules engine functioning in parallel to the aforementioned dialog model to facilitate diagnosis and filter protocols based on patient condition and attributes through a sequential process of computer generated questions deployed through the aforementioned audio device(s) and user responses that include the ability to request the system repeat a request or edit a previously entered patient attribute; and, a networked application for document generation of a digital and/or print report for long term data retention.

2. The system of claim 1, a hands-free system further comprising: the stationary device voice capturing device; a wireless receiver in data communication with the dialog model and networked software applications that connect to the databases; wherein the networked logic state rules engine applies data from the databases to sequentially transmit requests to the user through the stationary device.

3. The system of claim 1 further comprising: a mobile device voice capturing device; a wireless receiver in data communication with the dialog model and networked software applications that connect to the databases; wherein the networked logic state rules engine applies data from the databases to sequentially transmit requests to the user through the stationary device.

4. The system of claim 1, wherein the databases are on servers connected through a world-wide web internet system.

5. The system of claim 1, wherein the database is a remote computer located remotely from an operating theater in which the veterinary procedure is taking place.

6. The system of claim 1, wherein the dialog model is a remote computer located remotely from an operating theater in which the surgical procedure is taking place.

7. The system of claim 1, wherein the networked logic state rules engine is a remote computer located remotely from an operating theater in which the veterinary procedure is taking place.

8. The system of claim 1, wherein the data tracking and analytics is a remote computer located remotely from an operating theater in which the veterinary procedure is taking place.

9. The system of claim 1, wherein the Hospital Information System is a remote computer located remotely from an operating theater in which the veterinary procedure is taking place.

10. The system of claim 1, wherein the digital and print application is a remote computer located remotely from an operating theater in which the veterinary procedure is taking place.

11. The system of claim 1, wherein the stationary device is a wireless voice-based personal assistant or similar device.

12. The system of claim 1, wherein the portable device is a wireless smartphone.

13. The system of claim 1, wherein the portable device is a wireless smartphone with a wireless microphone and earphone.

14. The system of claim 1, wherein the portable device is a wireless digital tablet.

15. The system of claim 1, wherein the portable device is a wireless digital tablet with a wireless microphone and earphone.

16. The system of claim 1, wherein the portable device is a tablet.

17. The system of claim 1, wherein the portable device is a laptop computer with a microphone and speakers.

18. The system of claim 1, wherein the portable device is a laptop computer with wireless microphone and earphone.

19. The system of claim 1, wherein the computer application is a sequential method for generating requests for patient attributes.

20. The system of claim 1, wherein the computer application is a sequential method for generating requests for patient attributes.

21. The system of claim 1, wherein the computer application is a sequential method for receiving responses from the user.

22. The system of claim 1, wherein the stationary device speaker is a part of the device.

23. The system of claim 1, wherein the stationary device speaker is wirelessly connected to the device.

24. The system of claim 1, wherein the system generated protocol recommendation is specific to the attributes entered by the user during the veterinary procedure.

25. The system of claim 1, wherein the system generated protocol recommendation, if accepted by the user, updates the patient database, HIS information, pharmaceutical inventory, and user activity tracking system.

26. The system of claim 1, wherein the system generated protocol recommendation, if not accepted by the user, updates the user activity tracking system.

27. The system of claim 1, wherein the patient attributes are stored in the patient session database so the user can stop a procedure or stop updating the patient attributes and return to the exact attribute request when the user starts the system.
